(19) United States
(12) Patent Application Publication
(10) Pub. No.: US 2008/0269578 A1
(43) Pub. Date: Oct. 30, 2008

(54) Method for Detecting an Analyte Within the Body of a Patient or an Animal

(75) Inventors: Annelies Goris, Eindhoven (NL); Rieko Verhagen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS , N.V., EINDHOVEN (NL)

(21) Appl. No.: 11/994,446

(22) PCT Filed: Jun. 30, 2006
(86) PCT No.: PCT/IB06/52194

§ 371 Date: Jan. 2, 2008

(30) Foreign Application Priority Data
Jul. 6, 2005 (EP) 05300564.1

(51) Int. Cl. A61B 5/1455

(52) U.S. Cl. 600/322; 600/310

(57) ABSTRACT

The invention relates to a method for detecting an analyte within tissue of a patient or an animal. In the laboratory medicine it is often necessary to determine the amount of analytes within body fluids such as blood. For that purpose a blood withdrawal is carried out by experienced medical personnel, and the blood is analysed in the laboratory. This ex-vivo measurement of analytes is uncomfortable for the patient. Moreover, there is an unwanted delay between the blood withdrawal and the time at which the measurement values are obtained from the laboratory. In order to avoid these disadvantages it is suggested to detect analytes 1 in the body fluids by using laser-induced breakdown spectroscopy (LIBS). In this way it is possible to generate a plasma 6 within tissue, e.g. skin 3, of the body 2 in a certain depth below the surface of the skin 3, to detect the plasma light 16, and to extract the desired information from an analysis of the detected plasma light. This method can be carried out by the patient at home making it more acceptable to him or her.

The invention refers to a method for detecting an analyte within the body of a patient or an animal, whereby the analyte is contained in body tissue.

An important first step when carrying out a diagnostic method for curing a patient or an animal is the acquisition of measurement values. A typical second step is the comparison of the measurement values with normal values in order to record any significant deviation. In a third step medical personnel attempts to attribute the deviation to a particular clinical picture in order to take the appropriate steps to cure the patient. This invention exclusively refers to the first step mentioned above.

The above-mentioned measurement values are often obtained from body fluids such as blood, urine or saliva. Body fluids contain electrolytes, which, in the case of illness, show a composition, which deviates from the composition encountered when the patient is healthy.

If the body fluid is blood it is collected from the patient by blood withdrawal, analysed ex-vivo in the laboratory, and the result is communicated to the general practitioner in order to take the appropriate steps. Blood withdrawal is a task to be performed by experienced medical personnel in order to minimize the risk for the patient. The insertion of a cannula into the person's vascular system might be an everyday task for physicians that has to be performed with high accuracy and care. Therefore medical personnel has to be highly skilled for tasks such as blood withdrawal. The physician has to find the appropriate blood vessel and has to introduce the distal end of the cannula with a high precision in order to prevent the generation of hematoma or effusions. Depending on the vascular system of the patient even a highly skilled and experienced physician may require several attempts to insert a needle into the blood vessel. Multiple attempts of puncturing are however painful and cause appreciable discomfort for the patient. This is why blood withdrawal is un-liked by many patients and is regarded to be unpleasant.

Moreover, such multiple attempts to puncture a blood vessel are also rather time intensive which is disadvantageous especially in emergency situations.

A disadvantage of the above method of obtaining measurement values from body fluids such as the human blood is the long delay between the time of measurement and the time at which the result is obtained. This delay is not always acceptable. In quite a few cases a timely treatment is necessary, especially in the case of arrhythmias. Other disadvantages are the invasiveness of the method and the associated risk of infection.

Laser-induced breakdown spectroscopy (LIBS), also known as laser plasma spectroscopy or laser-induced plasma spectroscopy, is a well-known technique for performing both qualitative and quantitative elemental analysis of materials and compounds. LIBS may be used more or less effectively for procuring elemental analysis of many different substances including compounds in the form of gases, liquids or solids. In accordance with a basic technique, which is known to the man skilled in the prior art, the light output from a pulsed laser is focused onto the surface of an object. Assuming the focused laser pulse has sufficient intensity, a small amount of material at the surface of the object is vaporized forming an high-temperature plasma consisting of ions and excited atoms that emit a particular spectrum of light which corresponds to the elemental constituents of the vaporized material. The elemental composition of the irradiated material may then be accurately determined through spectral analysis of the radiation emitted by the plasma. Multiple plasma generating laser pulses are often used in succession in order to obtain additional spectral data to improve the accuracy of the analysis.

The Spanish patent application ES 2 170 022 discloses the quantitative elemental analysis of liquids, inter alia of liquids of biological type such as urine or blood. The elemental analysis is carried out by using LIBS. The authors write in the introductory part of the patent application that it had been difficult in the prior art to find appropriate laser parameters in order to carry out LIBS in the case of liquids. They circumvent this problem by a rapid freezing of the liquid, for example with liquid nitrogen. In an ex-vivo measurement laser light having a wavelength of 532 nm is directed on the surface of the frozen sample which causes an ablation. The ablated material is transferred into the plasma state and is analysed.

It is an object of the invention to provide a non-invasive method for the detection of analytes within the body of a patient or an animal, in particular of analytes in body fluids. The method should show a particularly short delay between the time of measurement and the time the data are obtained.

This object and other objects are solved by the features of the independent claims. Further embodiments of the invention are described by the features of the dependent claims. It should be emphasized that any reference signs in the claims shall not be construed as limiting the scope of the invention.

The object is solved by a method for detecting at least one analyte within tissue of a patient or an animal by means of LIBS, whereby the method comprises the following steps:
a) exposing tissue (e.g. gum, mucosa, skin) of a patient or animal with electromagnetic radiation,
b) whereby the electromagnetic radiation is chosen to have a wavelength (λ) which is substantially transmitted by the outmost layer of the tissue, and
c) whereby the intensity (I) of the electromagnetic radiation is chosen to generate a plasma below the outmost layer of the tissue,
d) detecting the electromagnetic radiation emitted by the plasma,
e) identifying at least one spectral line characteristic of the analyte.

As can be derived from the last paragraph analytes are detected within tissue of the patient or the animal, dead or alive, that is below the outmost layer of the tissue. The exposed tissue can be located on the outside the body of the patient or animal and might be the skin, e.g. the skin of the arm or the leg. The exposed tissue can also be located within the body, and might be accessed by body opening such as the mouth, the anus, the nose, or the ear. Furthermore, arbitrary butchered parts of animals can be exposed by the radiation for investigation purposes. The following description will for illustrative reasons primarily refer to the case of an investigation of the skin of a human patient, but the man skilled in the art will understand that the invention is not restricted to this case.

The outmost layer of a skin, for example the epidermis in the case of human beings, is not damaged with this approach as there is no laser ablation. This means that the method is non-destructive for the epidermis and generally non-invasive in nature, rendering it more acceptable for the patient while at the same time reducing the risk of infections.

The used radiation is not absorbable by the epidermis in order to ensure that the plasma volume lies within the body and below the epidermis. As only a negligible body volume is vaporized and transferred into the plasma state, that means that only a limited amount of energy is transferred from the plasma region to the epidermis by means of thermal conductivity. The patient will thus not suffer heat-induced pains, such that the method is easily accepted by the patient.

Another advantage of the suggested method is the quasi-instantaneous acquisition of measurement values having a particularly small delay between the time of measurement and the time at which the measurement values are obtained. The reason is as follows: The plasma is short lived and exists for a few nanoseconds. The acquisition of the electromagnetic spectrum of the plasma occurs in the same time interval. This means that the time which is needed to get measurement values is roughly equal to the time for processing and analysing the electromagnetic spectrum with electronic circuitry and/or computer program means. Depending on the capabilities on the processing unit chosen for that purpose the acquisition only takes less than few seconds.

It has to be emphasized that the measurement values which are obtained with the LIBS method concern the existence of chemical elements as the generated plasma destroys all chemical bonds. It is thus not possible to detect the existence of chemical compounds such as proteins with LIBS, at least not directly.

The used method is a very sensitive method, whereby the sensitivity strongly depends on the chemical element. The sensitivity is in almost all cases smaller than 500 ppm, and in many cases smaller than 100 ppm.

Another advantage of the method described therein is that LIBS represents a single method for the simultaneous measurement of analytes. This means that a single method is used in order to detect a plurality of analytes at the same time. In other words there is no need to use different techniques in order to detect different analytes.

A further advantage of the method being used is that the detection of analytes can be performed qualitatively and quantitatively. It is thus possible to get a qualitative result, for example that tissue is contaminated with arsen (As). In the same fashion it is possible to get a quantitative result, e.g. to detect a contamination with unwanted chemical elements such as mercury. The World Health Organization (WHO) for example suggests that most kinds of fish should contain less than 0.5 mg per kg. The exceeding of this threshold value which can be easily checked by the suggested method. This demonstrates that the method can be used in food industry for checking the existence of unwanted analytes.

Another advantage or the suggested method is that it can be carried out with an apparatus which is very compact. Taken into account the miniaturization of electronic components it is possible without too much effort to design a portable apparatus such as a table top device. The size is then roughly half a shoe carton. It can be expected that with using an optimised design and by using particularly small components the apparatus can be further downscaled. The small size of the apparatus makes it possible to give it to the patient such that he or she can carry out the measurements at home. This is a very comfortable and flexible way of carrying out the method that needs no expensive medical personnel, thus saving money in an expensive health system. Furthermore, the time interval between measurements can be chosen to be shorter than in the case that the measurements have to be carried out in a surgery or a hospital. This is particularly valuable in cases when a medical problem turns up suddenly is not constant in nature, for example in the case of arrhythmias.

A first step of the claimed method consists in exposing tissue, in this case the skin of the patient or the animal, with electromagnetic radiation. This electromagnetic radiation, which will also be called light in the description which follows, is preferably the light of a laser. This light is directed to and focused in the skin. This can be done by conventional means such as mirrors and lenses or fiber optics.

The wavelength of the electromagnetic radiation is chosen in such a way that it is substantially not absorbed by the outmost layer of the tissue. The intention of this choice is that the outmost layer of the tissue, which in the case of the skin of the human patient is the epidermis, shall not be damaged in order to avoid a wound, bleeding or the like.

The focal point is chosen to be below the outmost layer of the skin. Its position is determined as follows. The minimum depth of the focal point below the skin surface is determined by the thickness of the epidermis. Thus the minimum depth below the skin surface is between 0.03 mm to 0.3 mm depending on the body portion where the measurement is carried out. Technically it is possible to choose a focal point, which is up to 4 mm below the skin surface. Depending on the structure and composition of the skin layers the depth of the focal point below the skin surface is restricted by the fact that the energy input into the epidermis should be below 3.5 J/cm² at 1064 nm wavelength. The reason is that a larger energy input increases the risk that the epidermis is thermally damaged and that the patient suffers pain from the treatment. Another aspect, which determines the depth of the focal point below the skin surface is whether the light is absorbed by melanin. If there is no melanin in the tissue the depth can be chosen to be larger than 4 mm, whereas in the case that there is melanin in the skin the depth should not exceed 4 mm.

The intensity of the electromagnetic radiation must be chosen to generate a plasma below the outmost layer of the skin. The intensity at the focal point depends on the wavelength and should exceed 5*10¹¹ W/cm² at 1000 nm wavelength. The laser energy being deposited in the proximity of the focal point vaporizes the tissue and transfers it into a plasma state. The hot plasma emits a radiation, also being called the plasma light, which is detected by appropriate detection means, in particular a spectrograph. The electromagnetic spectrum is processed by appropriate processing means which makes it possible to detect spectral lines within the spectrum being characteristic for chemical elements. If the apparatus for carrying out the method has been calibrated by means of compositions with known contents and known amounts a quantitative result is obtained.

In a preferred embodiment of the invention the investigated body fluids are tear-drops, saliva or most importantly blood. In the alternative the analyte is a constituent of the tissue, and even possibly of bones.

Blood as an electrolyte is of particular importance as many substances are transported by the blood to different parts of the body such that the chemical composition of blood is very often examined in the laboratory medicine. It is of particular value that the suggested method is able to detect elements within the blood in a non-invasive way by choosing a focal point within a blood vessel.

As explained above the laser light being chosen for the exposition of a patient or animal is chosen in such a way that it is substantially transparent for the outmost layer of the tissue/skin. For that purpose radiation in the near infrared region is used, preferably having wavelengths between 600 nm and 1200 nm, and particularly between 800 nm and 1200 nm. This wavelength range is also called the therapeutic window.

Of particular importance for the non-destructive nature of the method is that only a limited body volume is vaporized. For that purpose mechanical side effects which might increase this body volume should be kept to a minimum. This object is achieved by using pulsed electromagnetic radiation, e.g. a pulsed laser light, having pulses in the femtosecond to picosecond range. Longer pulses however, for example pulses well in the nanosecond range, generate high-energy plasmas which destroy surrounding tissue by means of the associated mechanical effects such as shockwaves and cavitation bubbles.

The method for detecting an analyte within tissue of a patient or animal by means of LIBS has a wide range of applications. A first possibility among many possibilities is that the method is applied for the detection and/or prediction of arrhythmias. In that case the method is of particular value for patients as they can control their health status at home and can contact a medical doctor or hospital if necessary. For the treatment of arrhythmias magnesium (Mg), potassium (K), sodium (Na) and possibly copper (Cu) and tin (Zn) are detected by LIBS.

Another application of the suggested method is the follow-up diuretic therapy of patients with heart failure. In this case a multitude of chemical elements might be detected by the method and can be used by the general practitioner to improve the treatment of the patient. Another area of application is the diagnosis of heart attacks or for the diagnosis of micronutrient deficiencies.

These and other aspects of the invention will be apparent from and elucidated with the reference to the embodiments described thereafter.

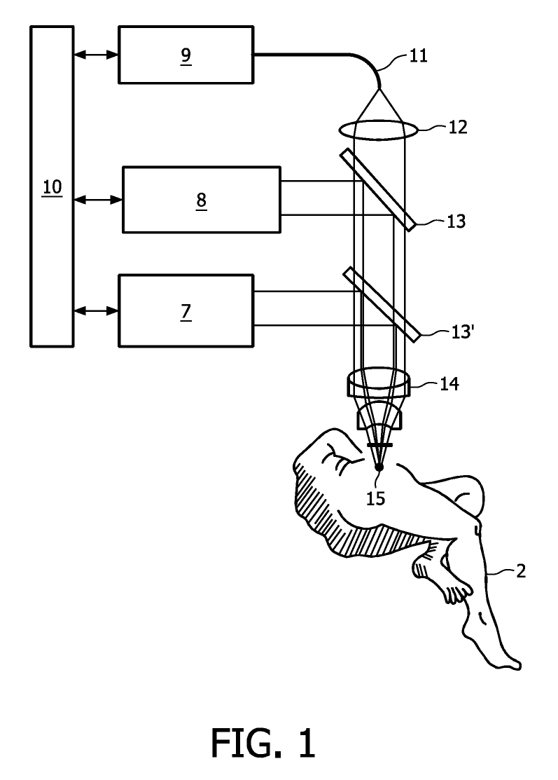

FIG. 1 shows in a schematic way an apparatus for carrying out the invention,

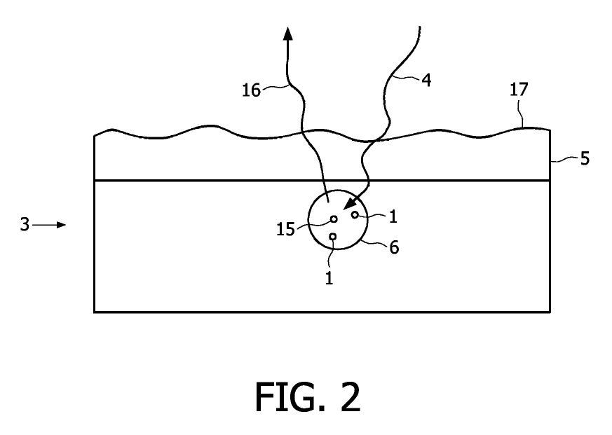

FIG. 2 shows a cut-out of the skin in a schematic way,

Table 1 shows a periodic table of elements and the typical detection limits for LIBS.

FIG. 1 shows in a very schematic way the apparatus for carrying out the method described above and a patient treated by the method. It should be emphasized that this FIG. 1 is not to scale, which also means that for illustrative purposes the size of the apparatus is exaggerated in comparison to the size of the body of the patient.

The apparatus for carrying out the method comprises a pulsed laser source 7 emitting light which is reflected by the dichroic beam splitter 13′ downwards to the patient 2. The laser light has a wavelength of 1064 nm and stems from an Nd:YAG laser whose laser light is focused by a high aperture laser objective 14 in a focal point 15 within the patient 2.

As will be discussed below with the help of FIG. 2, focal point 15 of the laser light is below the skin surface and generates a plasma. The detected plasma light is coupled into the apparatus and reaches a spectrograph 9 after passing the high aperture laser objective 14, the dichroic beam splitter 13′, the second dichroic beam splitter 13, the projection lens 12 and the optical fibre 11.

The spectrograph 9 is a conventional spectrograph of the size of a matchbox which separates the plasma light and detects its spectral components by a detection unit (not shown). The output of the detection unit is passed to the processing and control unit 9 in order to obtain the information which chemical elements were found in the plasma. The processing unit 10 contains calibration data, the calibration data stemming from probes with known amounts of chemical elements therein. With help of the calibration data it is not only possible to determine the existence of chemical elements within the plasma as such, but also to determine their amount.

The position where the plasma is generated can be controlled by a camera/imaging system 8 that observes the skin through the objective 14, allowing the laser 7 to pulse only when the appropriate target position is in focus.

It is possible to use means for manipulating the position of the target with respect to the laser focal point (not shown) such that the target within the body 2, for example a blood vessel, remains in the focus 15 during a longer period of time. This facilitates taking numerous measurements in order to obtain a time evolution of the concentration of various analytes or to increase the detection sensitivity by averaging the results of a number of plasma events.

In the embodiment shown in FIG. 1 various optical paths for the laser light, for the light reaching a spectrograph 9, and for the light reaching the camera 8 are spatially separated by means of the dichroic beam splitters 13, 13′. In the alternative it is possible to physically separate the various paths by using different elements for focusing, for collecting of plasma light, and for the imaging of the selected skin part.

The light emitted by laser 7 has a wavelength of 1064 nm with a repetition rate of 10 Hz generating a laser intensity of 5×10¹¹ W/cm² at the focal point 15. The focal point is chosen to lie within a blood vessel being roughly 0.6 mm below the skin surface. The method is used for a follow-up of diuretic therapy and patients with heart failure at home. For that purpose the existence and amount of Cu, Ca, Mg, Na, K and Zn in the blood is determined.

FIG. 2 shows in a schematic way a cut-out of the skin 3 with plasma 6 below the skin surface 17. The outmost layer of the skin 3, the epidermis 5, allows that the laser light 4 can be coupled into the body 2 without being absorbed significantly. The laser light 4 is focused below the epidermis 5 at a focal point 15 roughly 0.6 cm below the skin surface 17. The focal point 15 lies within a blood vessel (not shown). The laser light 4 creates a plasma 6. In this plasma 6 of very high temperature all the constituents of the blood are vaporized and torn apart such that the plasma only consists of atoms, ions and electrons. The detection of the plasma light 16 allows to detect analytes 1 within the plasma.

Table 1 shows a periodic table of elements and the typical detection limits for LIBS. As can be derived from this periodic table of elements the detection limits are nearly always below 500 ppm. This means that the method according to the invention is a very sensitive method with which a wide range of chemical elements can be detected with high accuracy.

TABLE 1
Periodic table of the elements

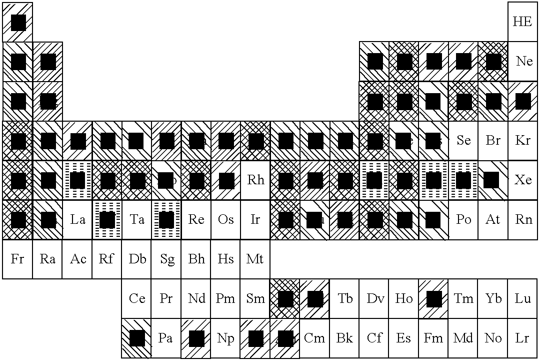

Typical detection limits for LIBS

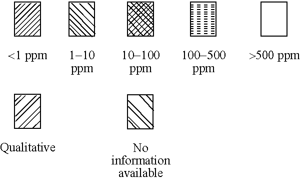

1. Method for detecting an analyte (1) within tissue of a patient (2) or of an animal by means of LIBS, the method comprising the following steps:
a) exposing tissue (3) of the patient or the animal with electromagnetic radiation (4),
b) the electromagnetic radiation having a wavelength such that it is transmitted by the outmost layer (5) of the skin,
c) the intensity of the electromagnetic radiation being chosen to generate a plasma (6) below the outmost layer of the skin,
d) detecting the electromagnetic radiation (16) emitted by the plasma,
e) identifying at least one spectral line characteristic of the analyte.

2. Method according to claim 1, characterized in that the analyte is contained in a body fluid within the tissue, and whereby the body fluid is preferably blood.

3. Method according to claim 1, characterized in that the electromagnetic radiation has a wavelength in the near infrared region.

4. Method according to claim 3, characterized in that the electromagnetic wavelength is between about 600 nm and 1200 nm, preferably between about 800 nm and 1200 nm.

5. Method according to claim 1, characterized in that the intensity is above 5×10¹¹ W/cm².

6. Method according to claim 1, characterized in that the pulse length is within the femtosecond to picosecond range.

7. Method according to claim 1, characterized in that it is applied for the detection and/or prediction of arrhythmias.

8. Method according to claim 1, characterized in that it is applied for diuretic therapy.

9. Method according to claim 1, characterized in that it is used for the diagnosis of heart attacks.

10. Method according to claim 1, characterized in that it is applied for the diagnosis of micronutrition deficiencies.

11. Use of an apparatus for a laser-induced breakdown spectroscopy (LIBS) for the in-vivo detection of an analyte in the body of a patient or an animal.

12. Use according to claim 11, characterized in that the analyte is contained in a body fluid, in particular in blood.
